(19) United States
(12) Patent Application Publication
(10) Pub. No.: US 2022/0218180 A1
(43) Pub. Date: Jul. 14, 2022

(54) ENDOSCOPE INSERTION CONTROL DEVICE, ENDOSCOPE INSERTION CONTROL METHOD, AND NON-TRANSITORY RECORDING MEDIUM IN WHICH ENDOSCOPE INSERTION CONTROL PROGRAM IS RECORDED

(71) Applicant: OLYMPUS CORPORATION, Tokyo (JP)

(72) Inventor: Hirokazu NISHIMURA, Tokyo (JP)

(73) Assignee: OLYMPUS CORPORATION, Tokyo (JP)

(21) Appl. No.: 17/709,868

(22) Filed: Mar. 31, 2022

Related U.S. Application Data
(63) Continuation of application No. PCT/JP2019/038751, filed on Oct. 1, 2019.

(51) Int. Cl. A61B 1/00; A61B 1/05; G06V 10/82; G06V 20/40

(52) U.S. Cl. CPC A61B 1/00006; G06V 20/40; G06V 10/82; A61B 1/05

(57) ABSTRACT

An endoscope insertion control device includes at least one processor including hardware, the processor is configured to acquire a plurality of images related to insertion of an endoscope and obtained in time series, classify an insertion state of the endoscope based on the plurality of images, and select a next operation related to insertion of the endoscope by referring to a result of the classification, and the processor relaxes a forward movement condition when the insertion state is classified as the insertion state of forward movement.

### CROSS REFERENCE TO RELATED APPLICATION

This application is a continuation application of PCT/JP2019/038751 filed on Oct. 1, 2019, the entire contents of which are incorporated herein by this reference.

### BACKGROUND OF THE INVENTION

### 1. Field of the Invention

The present invention relates to an endoscope insertion control device and an endoscope insertion control method that enable reliable and easy insertion of an endoscope insertion portion.

### 2. Description of the Related Art

A medical endoscope has been conventionally widely used with which an organ or the like at a deep part in a bent body cavity is observed with an elongated insertion portion being inserted into the body cavity without dissection of a body surface, and as necessary, for example, various medical operations and treatments are performed by using a treatment instrument inserted in a treatment instrument channel of an endoscope insertion portion.

To observe an organ or the like, a surgeon inserts the endoscope insertion portion into a body cavity by hand or the like. However, a tract in the body cavity into which the endoscope insertion portion is inserted has elasticity and becomes deflected by force pushing the endoscope insertion portion at, for example, a bend part of the tract, which prevents smooth forward movement of the endoscope insertion portion in some cases.

Thus, an insertion operation of an endoscope requires experience and skill. In particular, it is known that a procedure of endoscope insertion into a large intestine requires experience and skill because an intestinal canal has a long and complicated travelling shape, and it is difficult to perform insertion into a transverse colon and a sigmoid colon, which are not fixed to a body cavity and are movable, and distress may potentially be inflicted on a patient depending on an insertion situation of an endoscope. Thus, an endoscope bending device that facilitates an insertion operation is disclosed in Japanese Patent No. 3645223. In the disclosure, a center of a lumen is detected by detecting a dark part in an image from image pickup means provided at a distal end of an insertion portion, and a bending angle instruction value is calculated so that a bending portion provided at the insertion portion is directed to the center of the lumen.

Recently, an automatic insertion endoscope that enables automatic insertion of the endoscope into a lumen has been developed. With such an automatic insertion endoscope, an endoscope distal end portion can be directed to a forward movement direction based on, for example, detection of a lumen direction and can be moved forward. Accordingly, the endoscope insertion portion can reach, for example, a deep part of a large intestine.

However, in either case of insertion by a surgeon and insertion of an automatic insertion endoscope, an insertion state of an insertion portion does not necessarily constantly change nor move in accordance with an insertion operation intended by the surgeon for the insertion portion. For example, when an operation to insert (move forward) the insertion portion is performed, the insertion state of the insertion portion does not necessarily become a favorable forward movement state but, for example, only slight forward movement is made due to deflection of the endoscope, friction with an intestinal wall, or the like, or the distal end portion potentially moves away from a destination when what is called a stick phenomenon occurs at a splenic flexure.

### SUMMARY OF THE INVENTION

An endoscope insertion control device according to an aspect of the present invention includes at least one processor including hardware, the processor is configured to acquire a plurality of images related to insertion of an endoscope and obtained in time series, classify an insertion state of the endoscope based on the plurality of images, and select a next operation related to insertion of the endoscope by referring to a result of the classification, and the processor relaxes a forward movement condition when the insertion state is classified as the insertion state of forward movement.

An endoscope insertion control method according to an aspect of the present invention includes acquiring a plurality of images related to insertion of an endoscope and obtained in time series, classifying an insertion state of the endoscope based on the plurality of acquired images, and selecting a next operation related to insertion of the endoscope by referring to a result of the classification, and the selecting the next operation includes relaxing a forward movement condition when the insertion state is classified as the insertion state of forward movement.

A non-transitory recording medium in which an endoscope insertion control program is recorded according to an aspect of the present invention is a non-transitory recording medium in which a program is recorded, the program being configured to cause a computer to execute processing of acquiring a plurality of images related to insertion of an endoscope and obtained in time series, processing of classifying an insertion state of the endoscope based on the plurality of images, and processing of selecting a next operation related to insertion of the endoscope by referring to a result of the classification, and the processing of selecting the next operation is processing of relaxing a forward movement condition when the insertion state is classified as the insertion state of forward movement.

### BRIEF DESCRIPTION OF THE DRAWINGS

FIG. 1 is a block diagram illustrating an endoscope system including an endoscope insertion control device according to a first embodiment of the present invention;

FIG. 2A is a block diagram for description of a specific configuration of an endoscope system 1;

FIG. 2B is a diagram illustrating an example of a forward-backward movement mechanism;

FIG. 3A is an explanatory diagram for description of time-series images and labels added to the time-series images;

FIG. 3B is an explanatory diagram for description of time-series images and labels added to the time-series images:

FIG. 3C is an explanatory diagram for description of time-series images and labels added to the time-series images;

FIG. 3D is an explanatory diagram for description of time-series images and labels added to the time-series images;

FIG. 3E is an explanatory diagram for description of time-series images and labels added to the time-series images:

FIG. 3F is an explanatory diagram for description of time-series images and labels added to the time-series images;

FIG. 4 is a flowchart for description of operation of the embodiment;

FIG. 5 is a block diagram illustrating a second embodiment of the present invention;

FIG. 6 is an explanatory diagram illustrating a situation of an endoscope examination:

FIG. 7 is a flowchart for description of operation of the second embodiment; and

FIG. 8 is an explanatory diagram for description of the operation of the second embodiment.

### DETAILED DESCRIPTION OF THE PREFERRED EMBODIMENTS

Embodiments of the present invention will be described below in detail with reference to the accompanying drawings.

### First Embodiment

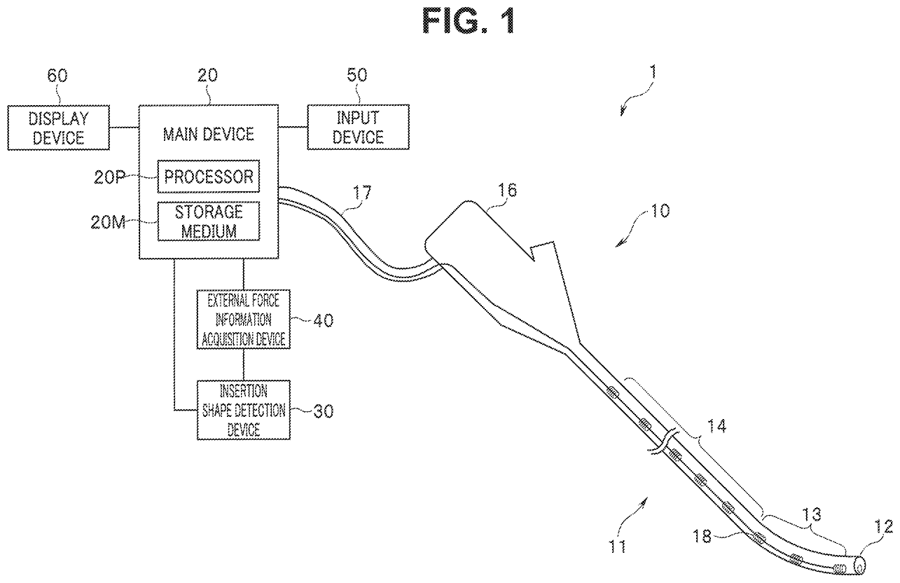

FIG. 1 is a block diagram illustrating an endoscope system including an endoscope insertion control device according to a first embodiment of the present invention. The first embodiment is an application to an automatic insertion endoscope configured to automatically insert an insertion portion of the endoscope into a large intestine of a subject.

In the present embodiment, whether an expected insertion state is obtained in response to an operation selected from among a plurality of kinds of operations for insertion of the endoscope insertion portion and which insertion state is obtained are determined by an identifier using a model acquired through machine learning. The identifier uses a model generated by producing teacher data through classification of each of a plurality of images (time-series images) obtained through continuous and time-sequential image pickup in accordance with an insertion state and performing learning of the teacher data by using a neural network. The identifier estimates a class in accordance with the insertion state based on the time-series images.

FIG. 1 is a diagram illustrating the configuration of a main part of the endoscope system including the endoscope insertion control device. For example, as illustrated in FIG. 1, an endoscope system 1 includes an endoscope 10, a main device 20, an insertion shape detection device 30, an external force information acquisition device 40, an input device 50, and a display device 60.

The endoscope 10 is an automatic insertion endoscope, insertion of which is automated as described later. The endoscope 10 includes an insertion portion 11 that is inserted into a subject, an operation portion 16 provided on a proximal end side of the insertion portion 11, and a universal cord 17 extended from the operation portion 16. The endoscope 10 is removably connected to the main device 20 through a scope connector (not illustrated) provided at an end part of the universal cord 17. In addition, a light guide (not illustrated) through which illumination light supplied from the main device 20 is transmitted is provided inside the insertion portion 11, the operation portion 16, and the universal cord 17.

The insertion portion 11 has flexibility and an elongated shape. The insertion portion 11 includes, sequentially from a distal end side, a distal end portion 12 that is rigid, a bending portion 13 that is formed freely bendable, and an elongated flexible tube portion 14 having flexibility. In addition, a plurality of source coils 18 configured to generate magnetic field in accordance with a coil drive signal supplied from the main device 20 are disposed at predetermined intervals in a longitudinal direction of the insertion portion 11 inside the distal end portion 12, the bending portion 13, and the flexible tube portion 14.

The distal end portion 12 is provided with an illumination window (not illustrated) through which the illumination light transmitted through the light guide provided inside the insertion portion 11 is emitted to an object. The distal end portion 12 is also provided with an image pickup unit 110 (not illustrated in FIG. 1) configured to perform operation in accordance with an image pickup control signal supplied from the main device 20, pick up an image of the object illuminated with the illumination light emitted through the illumination window, and output an image pickup signal.

The bending portion 13 can bend in accordance with control by a bending control unit 242 to be described later. The bending portion 13 can also bend in accordance with an operation of an angle knob (not illustrated) provided at the operation portion 16.

The operation portion 16 has a shape with which the operation portion 16 can be grasped and operated by a user. The operation portion 16 is provided with the angle knob with which it is possible to perform an operation to bend the bending portion 13 in, for example, four directions or eight directions in upward, downward, rightward, and leftward directions intersecting a longitudinal axis of the insertion portion 11. The operation portion 16 may also be provided with one or more scope switches (not illustrated) through which instruction can be performed in accordance with an input operation by the user.

The main device 20 includes at least one processor 20P and a storage medium 20M. The main device 20 is removably connected to the endoscope 10 through the universal cord 17. The main device 20 is also removably connected to the components of the insertion shape detection device 30, the input device 50, and the display device 60. The main device 20 performs operation in accordance with an instruction from the input device 50. The main device 20 generates an endoscope image based on an image pickup signal outputted from the endoscope 10 and performs operation of displaying the generated endoscope image on the display device 60. In addition, the main device 20 generates and outputs various kinds of control signals for controlling operation of the endoscope 10. The main device 20 has functions as an endoscope control device and performs control related to an insertion operation of the insertion portion 11 by using insertion shape information (to be described later) outputted from the insertion shape detection device 30. The main device 20 may generate an insertion shape image in accordance with the insertion shape information outputted from the insertion shape detection device 30 and perform operation for displaying the generated insertion shape image on the display device 60.

The insertion shape detection device 30 detects the magnetic field generated from each of source coils 18 provided in the insertion portion 11 and acquires each position of a plurality of source coils 18 based on intensity of the detected magnetic field. In addition, the insertion shape detection device 30 generates insertion shape information indicating the respective positions of the plurality of source coils 18 thus acquired and outputs the generated insertion shape information to the main device 20 and the external force information acquisition device 40. In other words, the insertion shape detection device 30 acquires insertion shape information by detecting an insertion shape of the insertion portion inserted in the subject and outputs the acquired insertion shape information to the main device 20 and the external force information acquisition device 40.

The external force information acquisition device 40 stores, for example, data of a curvature (or curvature radius) and a bending angle of the insertion portion 11 at a plurality of predetermined positions in a state in which no external force is applied, and data of the curvature (or curvature radius) and the bending angle at the plurality of predetermined positions, which is acquired in a state in which predetermined external force is applied at any position on the insertion portion 11 in every assumed direction. For example, the external force information acquisition device 40 specifies the respective positions of the plurality of source coils 18 provided in the insertion portion 11 based on the insertion shape information outputted from the insertion shape detection device 30, and acquires a magnitude and a direction of external force at the respective positions of the plurality of source coils 18 by referring to, based on the curvature (or curvature radius) and the bending angle at the respective positions of the plurality of source coils 18, various kinds of data stored in advance. In addition, the external force information acquisition device 40 generates external force information indicating the size and the direction of the external force at the respective positions of the plurality of source coils 18 thus acquired and outputs the generated external force information to the main device 20.

Note that, in the present embodiment, a method disclosed in Japanese Patent No. 5851204 or a method disclosed in Japanese Patent No. 5897092 may be used as a method by which the external force information acquisition device 40 calculates external force at the respective positions of the plurality of source coils 18 provided in the insertion portion 11. In the present embodiment, when the insertion portion 11 is provided with an electronic component such as a distortion sensor, a pressure sensor, an acceleration sensor, a gyro sensor, or a wireless element, the external force information acquisition device 40 may calculate external force at the respective positions of the plurality of source coils 18 based on a signal outputted from the electronic component.

The input device 50 includes at least one input interface operated by the user, such as a mouse, a keyboard, or a touch panel. The input device 50 can output, to the main device 20, an instruction in accordance with an operation by the user.

The display device 60 includes a liquid crystal monitor or the like. The display device 60 can display, on a screen, for example, an endoscope image outputted from the main device 20.

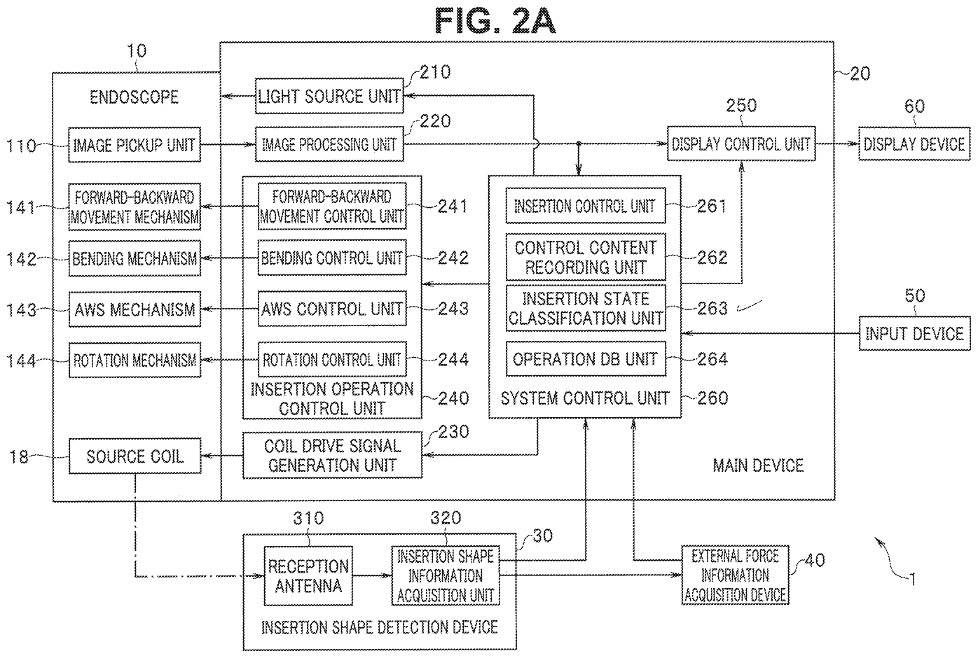

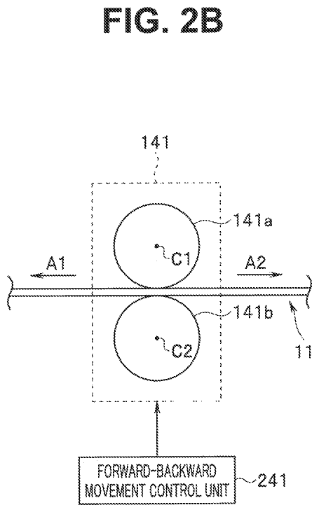

FIG. 2A is a block diagram for description of a specific configuration of the endoscope system 1. An example of specific configurations of the endoscope 10 and the main device 20 will be described below with reference to FIG. 2A.

The endoscope 10 includes the plurality of source coils 18, the image pickup unit 110, a forward-backward movement mechanism 141, a bending mechanism 142, an AWS (air feeding, water feeding, and suction) mechanism 143, and a rotation mechanism 144.

The image pickup unit 110 includes, for example, an observation window on which return light from an object illuminated with illumination light is incident, and an image sensor such as a color CCD configured to output an image pickup signal upon image pickup of the return light.

FIG. 2B is a diagram illustrating an example of a specific configuration of the forward-backward movement mechanism 141. The forward-backward movement mechanism 141 includes, for example, a pair of rollers 141a and 141b disposed at positions opposite to each other with the insertion portion 11 interposed between the rollers, and a non-illustrated motor configured to supply rotational drive force for rotating the pair of rollers 141a and 141b. For example, the forward-backward movement mechanism 141 can selectively perform either one of operation for moving forward the insertion portion 11 in an arrow A1 direction and operation for moving backward the insertion portion 11 in an arrow A2 direction, by driving the motor in accordance with a forward-backward movement control signal outputted from a forward-backward movement control unit 241 of the main device 20 and rotating the pair of rollers 141a and 141b about axes C1 and C2 in accordance with the rotational drive force supplied from the motor.

The bending mechanism 142 includes, for example, a plurality of bending pieces provided at the bending portion 13, a plurality of wires coupled to the plurality of bending pieces, and a motor configured to supply rotational drive force for pulling the plurality of wires. For example, the bending mechanism 142 can bend the bending portion 13 in the four directions of the upward, downward, rightward, and leftward directions by driving the motor in accordance with a bending control signal outputted from the main device 20 and changing a pulling amount of each of the plurality of wires in accordance with the rotational drive force supplied from the motor.

The AWS mechanism 143 includes, for example, two tracts of a non-illustrated air-water feeding tract and a suction tract provided inside the endoscope 10 (the insertion portion 11, the operation portion 16, and the universal cord 17), and an electromagnetic valve configured to perform operation of opening one of the two tracts and closing the other. For example, when the operation for opening the air-water feeding tract is performed by the electromagnetic valve in accordance with an AWS control signal outputted from the main device 20, the AWS mechanism 143 can circulate fluid including at least one of water or air supplied from the main device 20 to the air-water feeding tract and discharge the fluid through a discharge port formed at the distal end portion 12. For example, when the operation for opening the suction tract is performed by the electromagnetic valve in accordance with an AWS control signal outputted from the main device 20, the AWS mechanism 143 can exert suction force generated at the main device 20 on the suction tract so that any object near a suction port formed at the distal end portion 12 can be suctioned by the suction force.

The rotation mechanism 144 includes, for example, a grasping member that grasps the insertion portion 11 on the proximal end side of the flexible tube portion 14, and a motor configured to supply rotational drive force for rotating the grasping member. For example, the rotation mechanism 144 can rotate the insertion portion 11 about an insertion axis (longitudinal axis) by driving the motor in accordance with a rotation control signal outputted from the main device 20 and rotating the grasping member in accordance with the rotational drive force supplied from the motor.

As illustrated in FIG. 2A, the main device 20 includes a light source unit 210, an image processing unit 220, a coil drive signal generation unit 230, an insertion operation control unit 240, a display control unit 250, and a system control unit 260.

The light source unit 210 includes, for example, one or more LEDs or one or more lamps as light sources. The light source unit 210 can generate illumination light for illuminating inside of a subject into which the insertion portion 11 is inserted, and supply the illumination light to the endoscope 10. The light source unit 210 can change light quantity of the illumination light in accordance with a system control signal supplied from the system control unit 260.

The image processing unit 220 constituting an image acquisition unit together with the image pickup unit 110 includes, for example, an image processing circuit.

The image processing unit 220 generates an endoscope image by providing predetermined processing on an image pickup signal outputted from the endoscope 10 and outputs the generated endoscope image to the display control unit 250 and the system control unit 260.

The coil drive signal generation unit 230 includes, for example, a drive circuit. The coil drive signal generation unit 230 generates and outputs a coil drive signal for driving the source coils 18 in accordance with a system control signal supplied from the system control unit 260.

The insertion operation control unit 240 includes the forward-back ward movement control unit 241, the bending control unit 242, an AWS control unit 243, and a rotation control unit 244. The insertion operation control unit 240 performs operation for controlling a function achieved by the endoscope 10 based on an insertion control signal supplied from the system control unit 260. Specifically, the insertion operation control unit 240 performs operation for controlling at least one of a forward-backward movement function achieved by the forward-backward movement mechanism 141, a bending function achieved by the bending mechanism 142, an AWS function achieved by the AWS mechanism 143, or a rotation function achieved by the rotation mechanism 144.

The forward-backward movement control unit 241 generates and outputs, based on an insertion control signal supplied from the system control unit 260, a forward-backward movement control signal for controlling operation of the forward-backward movement mechanism 141. Specifically, the forward-backward movement control unit 241 generates and outputs, based on an insertion control signal supplied from the system control unit 260, for example, a forward-backward movement control signal for controlling a rotation state of the motor provided to the forward-backward movement mechanism 141.

The bending control unit 242 generates and outputs, based on an insertion control signal supplied from the system control unit 260, a bending control signal for controlling operation of the bending mechanism 142. Specifically, the bending control unit 242 generates and outputs, based on an insertion control signal supplied from the system control unit 260, for example, a bending control signal for controlling a rotation state of the motor provided to the bending mechanism 142.

The AWS control unit 243 can selectively perform either one of operation for supplying fluid including at least one of water or air to the endoscope 10 and operation for generating suction force for suctioning an object near the suction port of the distal end portion 12, by controlling a non-illustrated pump or the like based on an insertion control signal supplied from the system control unit 260. The AWS control unit 243 generates and outputs an AWS control signal for controlling operation of the AWS mechanism 143. Specifically, the AWS control unit 243 generates and outputs, based on an insertion control signal supplied from the system control unit 260, for example, an AWS control signal for controlling an operation state of the electromagnetic valve provided to the AWS mechanism 143.

The rotation control unit 244 generates and outputs, based on an insertion control signal supplied from the system control unit 260, a rotation control signal for controlling operation of the rotation mechanism 144. Specifically, the rotation control unit 244 generates and outputs, based on an insertion control signal supplied from the system control unit 260, for example, a rotation control signal for controlling a rotation state of the motor provided to the rotation mechanism 144.

In other words, based on an insertion control signal supplied from the system control unit 260, the insertion operation control unit 240 can generate and output, as a control signal corresponding to an operation achieved by a function of the endoscope 10, a control signal corresponding to each operation among a pushing operation as an operation for moving forward the insertion portion 11, a pulling operation as an operation for moving backward the insertion portion 11, an angling operation as an operation for bending the bending portion 13 to direct an orientation of the distal end portion 12 in directions (for example, one of eight directions, namely, the four directions of the upward, downward, rightward, and leftward directions and four middle directions between the four directions) intersecting the insertion axis (longitudinal axis) of the insertion portion 11, a twisting operation as an operation for rotating the insertion portion 11 about the insertion axis (longitudinal axis), an air feeding operation for ejecting gas in front of the distal end portion 12, a water feeding operation for ejecting liquid in front of the distal end portion 12, and a suction operation for suctioning a tissue or the like in front of the distal end portion 12.

The display control unit 250 performs processing for generating a display image including an endoscope image outputted from the image processing unit 220, and performs processing for displaying the generated display image on the display device 60. The display control unit 250 may also perform processing for displaying an insertion shape image outputted from the system control unit 260 on the display device 60.

As illustrated in FIG. 2A, the insertion shape detection device 30 includes a reception antenna 310 and an insertion shape information acquisition unit 320.

The reception antenna 310 includes, for example, a plurality of coils for three-dimensionally detecting the magnetic field generated by each of the plurality of source coils 18. The reception antenna 310 detects the magnetic field generated by each of the plurality of source coils 18, generates a magnetic field detection signal in accordance with the intensity of the detected magnetic field, and outputs the generated magnetic field detection signal to the insertion shape information acquisition unit 320.

The insertion shape information acquisition unit 320 acquires the respective positions of the plurality of source coils 18 based on a magnetic field detection signal outputted from the reception antenna 310. The insertion shape information acquisition unit 320 generates insertion shape information indicating the respective positions of the plurality of source coils 18 acquired as described above, and outputs the generated insertion shape information to the system control unit 260.

Specifically, the insertion shape information acquisition unit 320 acquires, as the respective positions of the plurality of source coils 18, for example, a plurality of three-dimensional coordinate values in a space coordinate system that is virtually set with an origin or a reference point at a predetermined position (such as an anus) on a subject into which the insertion portion 11 is inserted. The insertion shape information acquisition unit 320 generates insertion shape information including the plurality of three-dimensional coordinate values thus acquired, and outputs the generated insertion shape information to the system control unit 260. Then, in such a case, the system control unit 260 performs, for example, processing for acquiring a plurality of two-dimensional coordinate values corresponding to the plurality of respective three-dimensional coordinate values included in the insertion shape information outputted from the insertion shape information acquisition unit 320, processing for interpolating the plurality of acquired two-dimensional coordinate values, and processing for generating an insertion shape image in accordance with the plurality of interpolated two-dimensional coordinate values.

In the present embodiment, at least part of the insertion shape detection device 30 may be configured as an electronic circuit or may be configured as a circuit block of an integrated circuit such as a FPGA. In the present embodiment, for example, the insertion shape detection device 30 may include at least one processor (such as a CPU).

The system control unit 260 includes an insertion control unit 261, a control content recording unit 262, an insertion state classification unit 263, and an operation database (DB) unit 264. The system control unit 260 generates and outputs a system control signal for performing operation in accordance with instructions or the like from the operation portion 16 and the input device 50.

The insertion control unit 261 as an operation selection unit generates control signals (hereinafter referred to as basic control signals) for controlling various operations (hereinafter referred to as basic insertion operations) for inserting the insertion portion 11 into a desired lumen in accordance with automatic insertion control based on outputs from the insertion shape detection device 30, the external force information acquisition device 40, and the image processing unit 220.

A basic insertion operation by the insertion control unit 261 is selected and executed from among, for example, each of basic insertion operations achieved by functions of the endoscope 10 based on at least one of an endoscope image outputted from the image processing unit 220, external force information outputted from the external force information acquisition device 40, or an insertion shape image generated by the insertion shape detection device 30, and examples of the basic insertion operations include a forward movement operation (pushing operation), a backward movement operation (pulling operation), a stop operation, an angling (bending) operation, a rotational operation (twisting operation), an air feeding operation, a water feeding operation, and a suction operation as described above. The basic control signals from the insertion control unit 261 include control contents related to a moving amount, a moving speed, a rotational angle, a rotational direction, operation force, and the like when an insertion basic operation is executed.

The control content recording unit 262 is configured by a predetermined recording medium. The control content recording unit 262 sequentially records a basic insertion operation as a content of control by the insertion control unit 261.

In an insertion procedure for an endoscope having no automatic insertion function, an experienced and skilled doctor regards important a reaction on the distal end side in response to a hand-side operation on the endoscope. For example, when a distal end portion smoothly moves forward without resistance by an amount corresponding to a pushing amount, in other words, an amount intended by the doctor in response to a pushing operation with a right hand, the surgeon recognizes that the endoscope is in a favorable insertion state. In this case, it is easy for the surgeon to select a pushing operation intended for forward movement also in a next operation.

Such a state in which insertion is favorable is sometimes expressed as “the endoscope has a ‘free’ feel”, “the hand side and the distal end move in a one-to-one relation”, or the like. Hereinafter, such a favorable insertion state is referred to as a “state in which the endoscope is ‘free’”. When sufficient forward movement of the distal end portion relative to the pushing amount is not obtained, the distal end portion hardly moves, or motion different from forward movement, such as backward movement or rotation, occurs, the surgeon determines that a forward movement obstructing factor such as friction with a mucous membrane or deflection occurs. In this case, the experienced and skilled doctor selects an operation for removing or reducing the obstructing factor, such as a rotational operation or a jiggling operation (repetition of minute forward and backward motion). The determination is mainly based on an image and a force amount or a force feeling transferred from the endoscope to the right hand, and the former is important, in particular.

For example, when there is a loop such as an a loop, the pushing operation with the right hand can be performed without much resistance, but force for forward movement is absorbed by a loop portion, and accordingly, the image pickup unit 110 obtains an image (movie) in which the distal end portion 12 hardly moves, or obtains a movie indicating operation other than forward movement, such as rotation in a small amount. The experienced and skilled doctor can determine, based on such a movie, an actual insertion state upon an operation.

Thus, the insertion state classification unit 263 employed in the present embodiment includes an identifier having completed learning so that determination processing equivalent to that by such an experienced and skilled doctor can be executed. The insertion state classification unit 263 classifies (in other words, infers) an insertion state of the insertion portion 11 based on a series of images obtained through image pickup by the image pickup unit 110 and provided by the image processing unit 220, and obtains a result of the classification. Note that the series of images are a plurality of images (time-series images) obtained in time series, such as a movie of a predetermined frame rate or still images obtained through continuous photographing. A plurality of images that are obtained in time series but do not change at all are still time-series images.

In the present embodiment, whether an image change corresponding to an intended insertion state is obtained in response to an executed basic insertion operation can be recognized by the classification at the insertion state classification unit 263, and an operation thought to be effective for smooth insertion based on a result of the recognition is selected as an operation to be continued next (hereinafter referred to as an auxiliary insertion operation).

Information of auxiliary insertion operations thought to be effective for smooth insertion is registered in the operation DB unit 264 based on a relation between a basic insertion operation and an insertion state obtained as a result of the classification. The insertion control unit 261 acquires an auxiliary insertion operation by referring to the operation DB unit 264 based on a basic insertion operation recorded in the control content recording unit 262 and an insertion-state classification result outputted from the insertion state classification unit 263, and outputs a control signal (hereinafter referred to as an auxiliary control signal) for achieving the acquired auxiliary insertion operation.

A specific example of a configuration of the insertion state classification unit 263 in the present embodiment will be described below.

For example, the insertion state classification unit 263 performs processing using an identifier produced by learning, by a learning method such as deep learning, 3D coupling coefficients (weights) in a 3D-CNN (convolutional neural network) corresponding to a multi-layer neural network including an input layer, one or more convolutional layers, and an output layer, and accordingly, obtains a result of classification that an insertion state classified based on time-series images outputted from the image processing unit 220 is classified into one of a plurality of kinds. The 3D-CNN is a method obtained by extending a CNN (convolutional neural network), which is widely used for normal (two-dimensional) image recognition and classification, to applications to a three-dimensional image (voxel image) and time-series images.

At identifier production, machine learning is performed by using teacher data including, for example, a series of image-pickup images (time-series images) similar to those generated by the image processing unit 220, and a label indicating a result of classification that an insertion state determined based on the time-series images is classified into one of a plurality of predetermined kinds. Each of the above-described plurality of predetermined kinds is set as, for example, a characteristic insertion state that contributes to determination on success or failure of a manually or automatically performed insertion operation of the insertion portion 11 among various insertion states that can be formed in a duration between start and end of insertion of the insertion portion 11 into a subject, or is set as a kind of success or failure of the operation.

Examples of kinds of insertion states include not only simple “forward movement” but also “favorable forward movement” and “stop” including operation success or failure. At production of teacher data, for example, work is performed for providing, to one time-series image, a label in accordance with a determination result when an experienced and skilled person visually determines a kind to which the insertion state of the insertion portion 11 belongs among a plurality of predetermined kinds based on the time-series image.

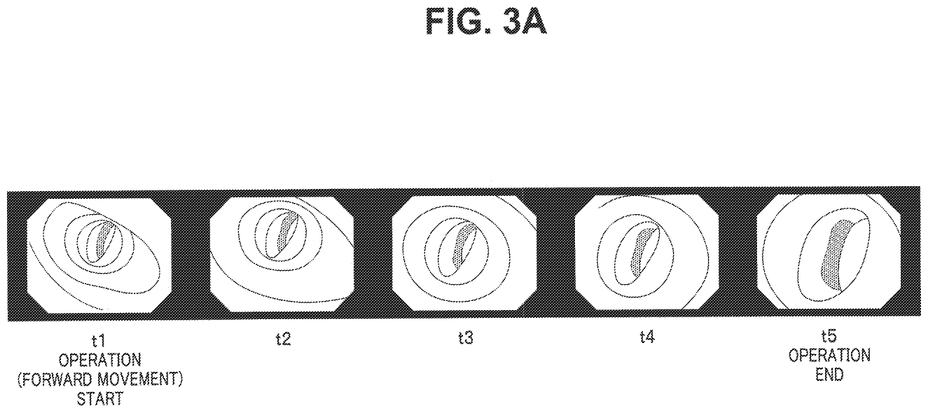

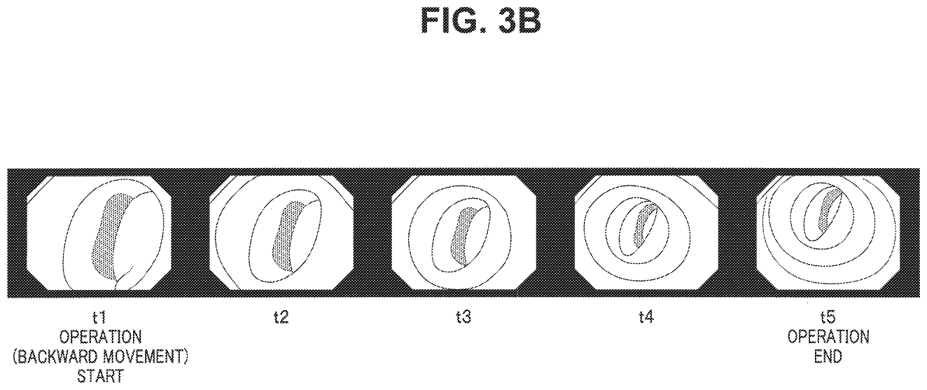

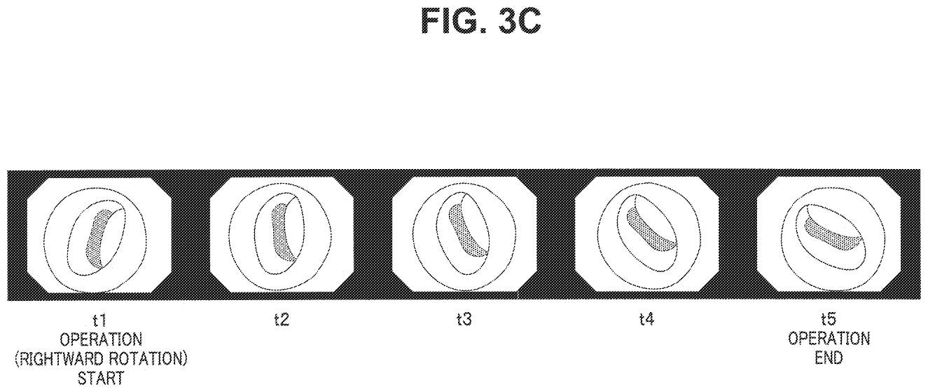

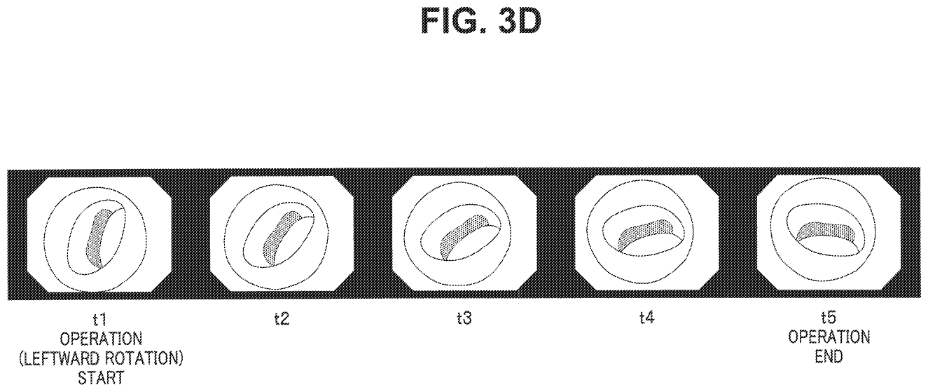

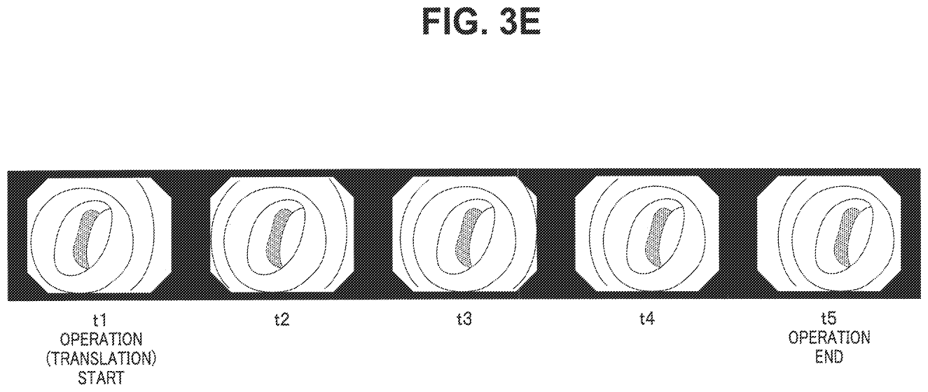

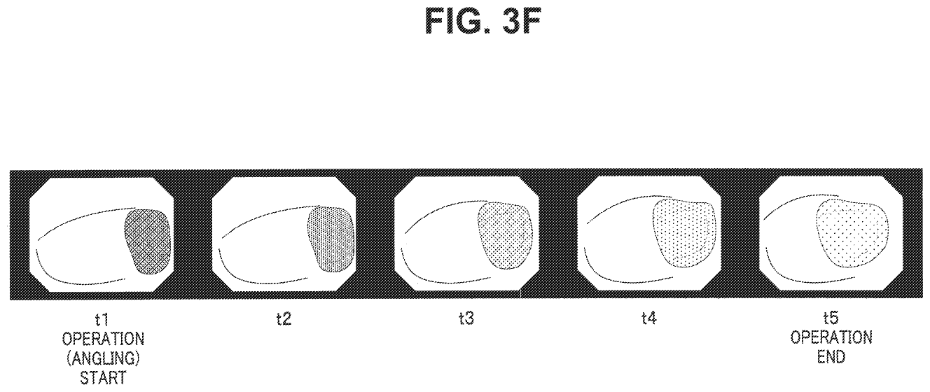

FIGS. 3A to 3F are explanatory diagrams for description of time-series images and labels added to the time-series images. FIGS. 3A to 3F each illustrate time-series images obtained through image pickup with the image pickup unit 110 at an insertion operation of the insertion portion 11, and time points t1, . . . , t5 indicate time points at which corresponding images in the time-series images are acquired. The time-series images can be acquired, for example, at constant time intervals from among a plurality of images obtained between start and end of an operation, and examples of the time-series images thus generated are illustrated in FIGS. 3A to 3F. At generation of time-series images, for example, when a wire or air pressure is used as a mechanism in an angling operation, operation of the insertion portion can slightly delay from an operation, and thus for example, an allowance time of one to two seconds approximately may be added to an end time point of the operation. Note that, in the present embodiment, description below assumes that a set of time-series images includes five images.

FIG. 3A illustrates time-series images in a case of a forward movement operation in which the distal end of the insertion portion 11 travels (moves forward) in a depth direction of a lumen. Such change that dark parts and folds of the lumen existing on a far side gradually come closer or move out of an image visual field as the distal end portion 12 moves forward appears in each of images sampled on the time axis.

In the present embodiment, it is assumed that a teacher data producer is an experienced and skilled doctor of a large-intestine endoscope insertion procedure or is a person having sufficient knowledge about an endoscope operation equivalent to knowledge of the experienced and skilled doctor.

For example, the teacher data producer provides a label “favorable forward movement” to each time-series image. The label “favorable forward movement” indicates, for example, an insertion state in which the distal end of the insertion portion 11 moves forward by 5 cm approximately when an operation to move forward the insertion portion 11 by 5 cm is performed. Note that, for example, a label “insufficient forward movement” may be provided to an insertion state in which the distal end of the insertion portion 11 moves forward by 2.5 cm approximately when an operation to move forward the insertion portion 11 by 5 cm is performed. For example, a label “stop” may be provided to an insertion state in which the distal end of the insertion portion 11 moves forward by an amount of 1 cm or less when an operation to move forward the insertion portion 11 by 5 cm is performed.

Note that although a forward movement amount for an operation is quantitatively expressed in the above description, an actual forward movement amount may be subjectively evaluated. For example, time-series images obtained when the forward-backward movement mechanism 141 performs an operation to move forward the insertion portion 11 by 5 cm may be provided with a label “favorable forward movement” when the teacher data producer evaluates that the forward movement is sufficient, a label “stop” in a case of a state in which almost no movement is made, or a label “insufficient forward movement” in a case of a forward movement amount that is felt as an intermediate different from any of the above-described labels.

Such classification of behavior of the endoscope in response to an operation into one of a plurality of classes in accordance with a change amount of the behavior is also possible for backward movement and rotation, and more accurate classification is possible for an operation by setting, for example, “rightward rotation by less than 90° ” and “rightward rotation by 90° or more”.

FIG. 3B illustrates time-series images in a case of a backward movement operation in the state in which the endoscope is “free”, and the distal end of the insertion portion 11 moves backward with respect to the depth direction of the lumen. For example, the teacher data producer provides a label “backward movement” to the time-series images in FIG. 3B. FIG. 3C illustrates time-series images in a case of a rightward rotational operation in the state in which the endoscope is “free”, and the distal end of the insertion portion 11 rotates rightward with respect to the depth direction of the lumen. For example, the teacher data producer provides a label “rightward rotation” to the time-series images in FIG. 3C. FIG. 3D illustrates time-series images in a case of a leftward rotational operation in the state in which the endoscope is “free”, and the distal end of the insertion portion 11 rotates leftward with respect to the depth direction of the lumen. For example, the teacher data producer provides a label “leftward rotation” to the time-series images in FIG. 3D.

Note that the time-series images in FIGS. 3B to 3D described above correspond to the state in which the endoscope is “free”, but the time-series images in FIGS. 3B to 3D described above are provided with the labels “backward movement”. “rightward rotation”, and “leftward rotation” irrespective of whether the endoscope is “free”. The label “favorable forward movement” in FIG. 3A described above is provided based on the assumption that the teacher data producer knows which operation is performed when the time-series images are obtained.

FIG. 3E illustrates time-series images provided with a label “rightward translation” by the teacher data producer, and FIG. 3F illustrates time-series images provided with a label “rightward angling operation” by the teacher data producer. Note that only an angling operation to bend the bending portion 13 in the rightward direction as a predetermined one direction is illustrated as an example in FIG. 3F, but labels corresponding to the eight directions, respectively, may be provided by using, for example, time-series images when angling operations in the eight directions are performed.

In a case, an operation causes an insertion state in which the distal end of the insertion portion 11 causes deformation of an intestinal canal side. A label “intestinal canal side deformation” may be provided by using time-series images in such a case. Note that the number of kinds of intestinal canal side deformation is not limited to one.

In this manner, machine learning is performed by using teacher data obtained by labeling each time-series image in accordance with the kind of an insertion state, and as a result, an identifier configured to classify an insertion state is obtained. In the learning, for example, identification targets may be a total of 14 classes of favorable forward movement, insufficient forward movement, backward movement, two-directional rotation, angling operations in the eight directions, and stop, which indicates a state with substantially no movement, about 1000 sets of time-series images may be used for each class as teacher data, and the number of times (called epochs) of learning may be 100. With such an identifier, for example, a plurality of likelihoods corresponding to respective kinds that can be classified as the kind of the insertion state of the insertion portion 11, which is obtained from time-series images outputted from the image processing unit 220 can be acquired as output data outputted from the output layer of a neural network by acquiring multi-dimensional data such as pixel values of each pixel included in the time-series images and inputting the multi-dimensional data as input data into the input layer of the neural network. Through processing using the identifier, for example, the kind of one insertion state corresponding to one highest likelihood among the plurality of likelihoods included in the output data outputted from the output layer of the neural network can be obtained as a classification result of the insertion state of the insertion portion 11, which is obtained as a result of an operation.

In other words, the insertion state classification unit 263 obtains a classification result indicating the kind of the insertion state of the insertion portion 11 as a result of an insertion operation into a subject by performing processing using an identifier produced through machine learning with teacher data including time-series images from the image processing unit 220 and a label indicating a result of classification that the insertion state of the insertion portion 11, which is obtained from the time-series images, is classified into one of a plurality of predetermined kinds.

As described above, the insertion control unit 261 reads an insertion state obtained as a result of classification from the insertion state classification unit 263, reads a basic insertion operation that causes the insertion state from the control content recording unit 262, and acquires an auxiliary insertion operation by referring to the operation DB unit 264. Note that when an insertion state expected for the basic insertion operation is obtained as in the state in which the endoscope is “free”, normally, no auxiliary insertion operation is performed but a next basic insertion operation is performed.

Note that, in the present embodiment, at least some of functions of the main device 20 may be achieved by the processor 20P. In the present embodiment, at least part of the main device 20 may be configured as an individual electronic circuit or a circuit block of an integrated circuit such as a FPGA (field programmable gate array). Configurations according to the present embodiment may be modified as appropriate so that, for example, a computer may read a program for executing at least some of the functions of the main device 20 from the storage medium 20M such as a memory and perform operation in accordance with the read program.

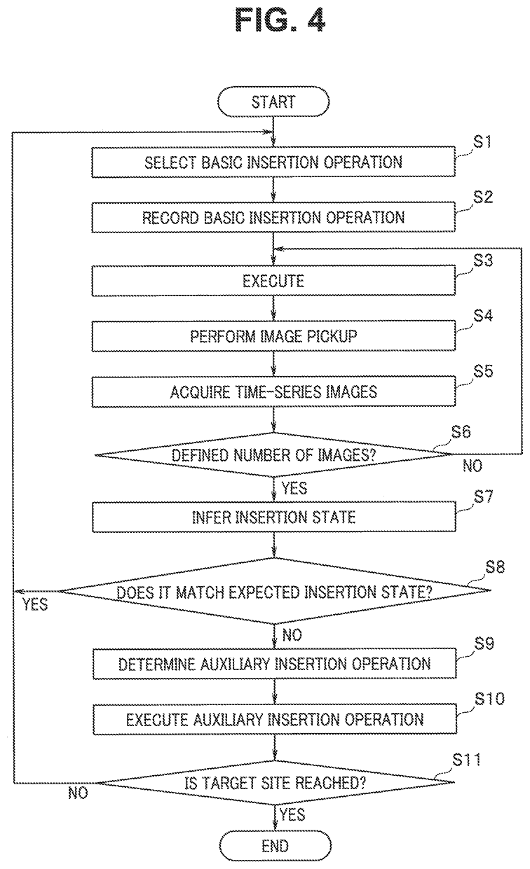

Subsequently, operation of the embodiment thus configured will be described below with reference to FIG. 4. FIG. 4 is a flowchart for description of the operation of the embodiment.

At step S1 in FIG. 4, the system control unit 260 selects a basic insertion operation. The system control unit 260 records contents of the selected basic insertion operation in the control content recording unit 262 (step S2). The insertion control unit 261 outputs a basic control signal for executing the selected basic insertion operation to the insertion operation control unit 240. Accordingly, the insertion operation control unit 240 executes the basic insertion operation by controlling each mechanism of the endoscope 10 (step S3).

The image pickup unit 110 picks up an image of a subject at insertion (step S4) and outputs an image pickup signal to the image processing unit 220. The image processing unit 220 provides an image-pickup image (endoscope image) based on the image pickup signal to the display control unit 250 to display the image on the display device 60 and also provides the image to the insertion state classification unit 263. The image processing unit 220 sequentially provides images picked up by the image pickup unit 110 to the insertion state classification unit 263, and accordingly, the insertion state classification unit 263 acquires time-series images in accordance with an insertion state of the distal end of the image pickup unit 110 in the basic insertion operation.

Note that, at step S6, the system control unit 260 determines whether a defined number of images is reached, and returns the processing to step S3 when the defined number of images is not reached, or advances the processing to step S7 when the defined number of images is reached. Accordingly, at step S7, the insertion state classification unit 263 classifies an insertion state by using time-series images constituted by the defined number of image-pickup images. The classification indicates an insertion state to which an image change caused by an operation related to insertion of the endoscope 10 corresponds.

For example, motion of the distal end portion 12, such as “favorable movement of the distal end portion”, “insufficient movement”, “no movement”, “backward movement (retraction of the lumen)”, or “rotation” can be acquired for an operation to move forward the distal end portion 12 by classification based on a plurality of time-series images picked up for the operation.

The insertion control unit 261 reads, from the control content recording unit 262, contents of a basic insertion operation that causes the insertion state obtained as a result of the classification, refers to the operation DB unit 264 based on the insertion state acquired by the classification and the read basic insertion operation, and then executes a next auxiliary insertion operation. In this case, the insertion control unit 261 determines whether the read basic insertion operation and the insertion state acquired by the classification correspond to each other, in other words, whether an expected insertion state is obtained (step S8). When the expected insertion state is obtained, the insertion control unit 261 returns the processing to step S1 to select a next basic insertion operation, or when the expected insertion state is not obtained, the insertion control unit 261 determines an auxiliary insertion operation at step S9.

For example, when a determination result other than “favorable movement of the distal end portion” is obtained for an operation to move forward the distal end portion 12, an operation such as jiggling or rotational operation for improving a situation so that the distal end portion 12 of the endoscope 10 can be smoothly moved is selected and executed as the auxiliary insertion operation (step S10).

For example, deflection removal or jiggling is selected for a determination result “no movement” so that movement is made. For example, for a determination result of “slight movement”, the next operation is selected or an operation to move forward with rotational force or the like is selected. For example, for a determination result “movement”, the normal next operation may be selected, the forward movement amount may be increased, or a forward movement condition may be relaxed, for example, a condition that forward movement is made even when the lumen is somehow off a visual field center may be selected.

The system control unit 260 determines whether the distal end portion 12 has reached a target site (step S11), and repeats the processing at steps S1 to S10 until the target site is reached.

In the present embodiment as described above, whether an expected insertion state is obtained in response to an operation selected from among a plurality of kinds of operations for insertion of the endoscope insertion portion and which insertion state is obtained can be determined by using an identifier configured to perform classification of time-series images in accordance with the insertion state of the endoscope distal end portion into a lumen. By determining a next insertion operation by using a result of the determination, it is possible to select an appropriate operation in accordance with a situation based on understanding of behaviors of the endoscope insertion portion and the distal end portion in response to an intended operation, and thus it is possible to more reliably and smoothly insert the endoscope insertion portion into the lumen without deterioration of an insertion situation nor pain of the patient. Moreover, the identifier can obtain a highly accurate classification result by using a model generated by producing teacher data through classification of time-series images in accordance with the insertion state of the endoscope distal end portion into the lumen and performing learning of the teacher data by using a neural network.

### Second Embodiment

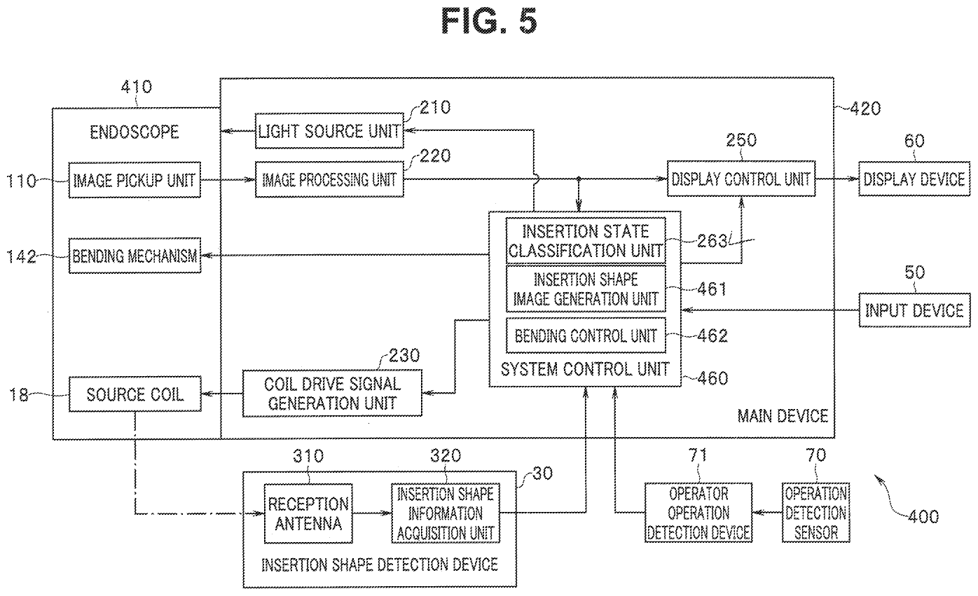

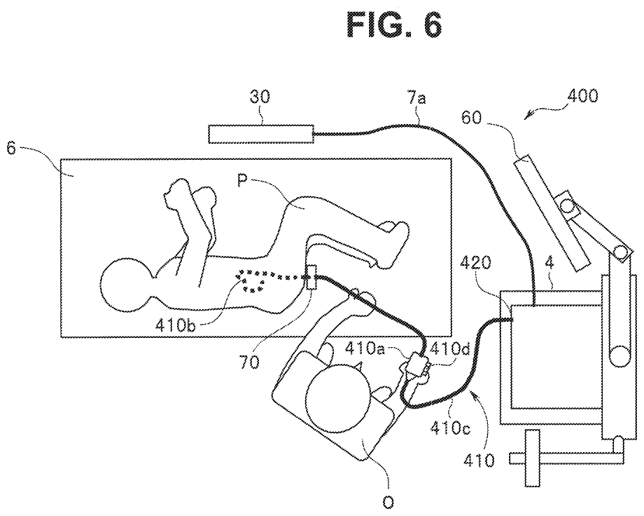

FIG. 5 is a block diagram illustrating a second embodiment of the present invention. FIG. 6 is an explanatory diagram illustrating a situation of an endoscope examination. In FIGS. 5 and 6, any component identical to a component in FIG. 2A is denoted by the same reference sign, and description of the component will be omitted.

In the first embodiment, an example of application to an automatic insertion endoscope is described. The present embodiment is an application to a typical endoscope, an insertion portion of which is inserted into a subject by a surgeon.

In FIGS. 5 and 6, an endoscope system 400 includes an endoscope 410 and a main device 420. As illustrated in FIG. 5, the endoscope 410 does not include the forward-backward movement mechanism 141, the AWS mechanism 143, and the rotation mechanism 144 of the endoscope 10 in FIG. 2A. The main device 420 does not include the insertion operation control unit 240 of the main device 20 in FIG. 2A. The main device 420 employs, in place of the system control unit 260 in FIG. 2A, a system control unit 460 not including the insertion control unit 261, the control content recording unit 262, and the operation DB unit 264 but additionally including an insertion shape image generation unit 461 and a bending control unit 462.

The endoscope 410 includes an elongated flexible insertion portion 410b that is inserted into a body cavity of a subject P, an operation portion 410a connected to a proximal end of the insertion portion 410b and provided with various operation units, and a cable 410c for connecting the operation portion 410a and the main device 420.

FIG. 6 illustrates a state in which the insertion portion 410b is inserted in a large intestine of the subject P laid on an examination bed 6 through an anus. FIG. 6 illustrates a situation in which a surgeon O grasps the operation portion 410a and the insertion portion 410b of the endoscope 410 connected to the main device 420 on a medical trolley 4 through the cable 410c.

A configuration of the insertion portion 410b is similar to a configuration of the insertion portion 11 in FIG. 1, in which the image pickup unit 110 is disposed at the distal end portion and the plurality of source coils 18 for insertion state detection are disposed. In addition, a bending portion is provided at a distal end of the insertion portion 410b and is configured to be driven to bend by the bending mechanism 142.

A bending knob 410d included in the input device 50 is disposed at the operation portion 410a. As the bending knob 410d is operated, an operation signal is supplied to the system control unit 460. The bending control unit 462 of the system control unit 460 generates and outputs, based on the operation of the bending knob 410d, a bending control signal for controlling operation of the bending mechanism 142. With the bending control signal, for example, the rotation state of the motor provided to the bending mechanism 142 is controlled to perform bending operation in accordance with the operation of the bending knob 410d. Accordingly, the surgeon can bend the bending portion by operating the bending knob 410d and push the insertion portion 410b into the body cavity.

An insertion state of the insertion portion 410b is observed by the known insertion shape detection device 30. The insertion shape detection device 30 including the reception antenna 310 and the insertion shape information acquisition unit 320 is disposed near the bed 6. The insertion shape detection device 30 is connected to the main device 420 through a cable 7a. The insertion shape detection device 30 generates insertion shape information of the insertion portion 410b and outputs the generated insertion shape information to the system control unit 460.

An insertion operation by the surgeon O can be detected by an operation detection sensor 70. The operation detection sensor 70 detects a forward-backward movement direction, a moving amount, a rotational direction, and a rotational amount of the insertion portion 410b, for example, near the anus of the subject P. The operation detection sensor 70 outputs a result of the detection to a surgeon operation detection device 71.

The surgeon operation detection device 71 determines a start timing of each of various operations by the surgeon based on the result of the detection by the operation detection sensor 70. The surgeon operation detection device 71 also determines a kind of the operation by the surgeon based on the result of the detection by the operation detection sensor 70 in a predetermined duration since the start timing of the operation. Note that the predetermined duration is set in accordance with time-series images in learning for obtaining a model used by the insertion state classification unit 263. The surgeon operation detection device 71 outputs information of the kind and the start timing of the operation by the surgeon to the system control unit 460.

The insertion shape image generation unit 461 of the system control unit 460 executes, for example, processing for acquiring a plurality of two-dimensional coordinate values corresponding to a plurality of three-dimensional coordinate values, respectively, included in the insertion shape information outputted from the insertion shape information acquisition unit 320, processing for interpolating the plurality of acquired two-dimensional coordinate values, and processing for generating an insertion shape image in accordance with the plurality of interpolated two-dimensional coordinate values. The insertion shape image generation unit 461 outputs the generated insertion shape image to the display control unit 250. Accordingly, the display control unit 250 can display the insertion shape image on a display screen of the display device 60.

In the present embodiment, the system control unit 460 includes the insertion state classification unit 263 that uses a model acquired by learning similar to, for example, learning in the first embodiment. The system control unit 460 outputs, to the display control unit 250, information of a classification result acquired by classification at the insertion state classification unit 263 and indicating the kind of an insertion state in response to the operation of the surgeon detected by the surgeon operation detection device 71. Accordingly, the display control unit 250 displays a display indicating the kind of the insertion state on the display screen of the display device 60.

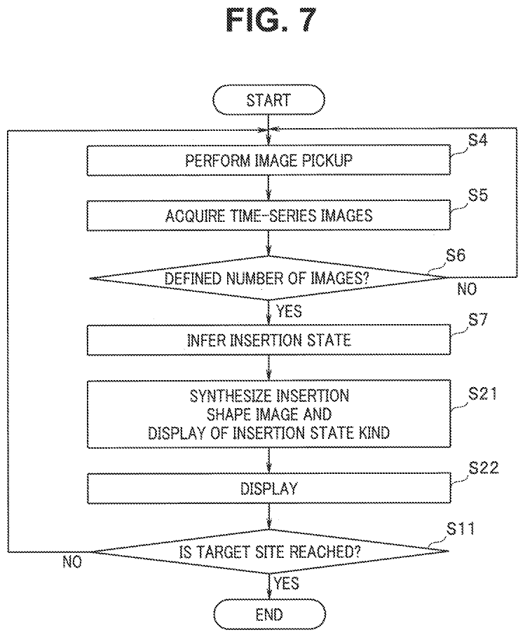

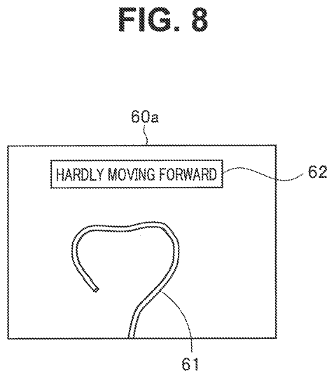

Subsequently, operation of the embodiment thus configured will be described below with reference to FIGS. 7 and 8. FIG. 7 is a flowchart for description of the operation of the second embodiment. In FIG. 7, any procedure identical to a procedure in FIG. 4 is denoted by the same reference sign, and description of the procedure will be omitted. FIG. 8 is an explanatory diagram for description of the operation of the second embodiment.

In the present embodiment, steps S1 to S3 in FIG. 4 are not performed in FIG. 7 since the endoscope 410 that cannot be automatically inserted is employed. At step S7 in FIG. 7, the insertion state classification unit 263 classifies an insertion state by using time-series images constituted by a defined number of image-pickup images. The classification indicates an insertion state to which an image change caused by a result of an insertion operation of the endoscope 10 performed by the surgeon corresponds. The insertion state classification unit 263 provides a result of the classification to the display control unit 250, and the display control unit 250 displays the result of the classification on the screen of the display device 60 (step S22).

FIG. 8 illustrates a display example in this case, and a display screen 60a of the display device 60 displays an insertion shape image 61 of the insertion portion 11, which is generated by the insertion shape image generation unit 461, and also displays a display 62 of the kind of the insertion state as the result of the classification. In the example illustrated in FIG. 8, the display 62 of the kind of the insertion state is a display “hardly moving forward”. The surgeon can easily recognize, based on the display 62, that the distal end of the insertion portion 11 hardly moves forward despite, for example, a forward movement operation.

In the present embodiment as described above, whether an expected insertion state is obtained in response to an operation selected from among a plurality of kinds of operations for insertion of the endoscope insertion portion and which insertion state is obtained can be determined by employing an identifier that uses a model generated by producing teacher data through classification of time-series images in accordance with the insertion state of the endoscope distal end portion into a lumen and performing learning of the teacher data by using a neural network. A result of the determination can be displayed to effectively support the surgeon for selection of a next insertion operation that can improve an insertion situation and prevent pain of the patient.

Note that the insertion operation control unit 240, the system control unit 260, the system control unit 460, and the like in the above-described embodiments may each be configured as a dedicated circuit or a combination of a plurality of general-purpose circuits, and as necessary, configured in combination with a processor such as a microprocessor or a CPU configured to perform operation in accordance with software programmed in advance, or with a sequencer. It may be designed such that part or all of control of the above-described configuration is performed by an external device, and in this case, a wired or wireless communication circuit is interposed. Characteristic processing and supplementary processing of each embodiment can conceivably be performed by an external instrument such as a server or a personal computer to form another embodiment. Thus, the present application includes a case in which characteristics of the present invention are achieved by a plurality of instruments in cooperation. Communication in this case employs Bluetooth (registered trademark), Wi-Fi (registered trademark), a phone line, or the like. Alternatively, the communication may be performed through a USB or the like. Dedicated circuits, general-purpose circuits, and control units may be integrated as an ASIC.

Most of technologies described above, mainly, controls and functions described with reference to flowcharts, can be set by a program and implemented as the program is read and executed by a computer. The program may be entirely or partially recorded or stored as a computer program product in a portable medium such as a flexible disk, a CD-ROM, or a non-volatile memory, or a storage medium such as a hard disk or a volatile memory, and may be distributed or provided through product shipment, a portable medium, or a communication line. A user can easily achieve the endoscope insertion control device according to the present embodiment by downloading the program through a communication network and installing the program on a computer or by installing the program on a computer from a recording medium.

The present invention is not limited to the above-described embodiments, but constituent components may be modified and materialized without departing from the gist of the present invention when the present invention is implemented. Various kinds of inventions may be formed by an appropriate combination of a plurality of constituent components disclosed in the above-described embodiments. For example, some of the constituent components indicated in the embodiments may be deleted. Moreover, constituent components in different embodiments may be combined as appropriate.

Note that although a case in which the 3D-CNN as a method of deep learning is used as a method of detecting a state of an endoscope insertion portion based on time-series images is described above, it is possible to employ various methods by which the same effects can be obtained in identification of time-series images, such as movie recognition using known optical flow.

The endoscope insertion control device and the method of the present invention are also applicable to an organ other than a large intestine, such as a small intestine or bronchi, and an industrial endoscope for performing an examination of a pipe or the like.

What is claimed is:

1. An endoscope insertion control device comprising at least one processor including hardware, wherein
the processor is configured to:
acquire a plurality of images related to insertion of an endoscope and obtained in time series;
classify an insertion state of the endoscope based on the plurality of images; and
select a next operation related to insertion of the endoscope by referring to a result of the classification, and
the processor relaxes a forward movement condition when the insertion state is classified as the insertion state of forward movement.

2. The endoscope insertion control device according to claim 1, wherein the processor:
selects an operation to move forward a distal end portion of the endoscope; and
classifies the insertion state of the endoscope according to the plurality of images in time series in accordance with execution of the operation to move forward the distal end portion.

3. The endoscope insertion control device according to claim 1, wherein, in an operation of relaxing the forward movement condition, the processor selects an operation to move forward even when a lumen is off a visual field center.

4. The endoscope insertion control device according to claim 1, wherein the processor classifies each of the plurality of images into any of a plurality of classifications related to motion of a distal end portion of the endoscope and/or motion of an insertion target body in which the endoscope is inserted.

5. The endoscope insertion control device according to claim 2, wherein the processor selects an operation based on whether the result of the classification is a classification generated when an operation selected before is appropriately executed.

6. The endoscope insertion control device according to claim 1, wherein
the processor outputs a classification result as either a first classification or a second classification, the first classification corresponding to sufficient forward movement of the endoscope when an operation related to forward movement is selected, the second classification corresponding to insufficient forward movement, and
changes a selected operation based on whether the classification result generated when the operation related to forward movement of the endoscope is selected is the first classification or the second classification.

7. The endoscope insertion control device according to claim 1, wherein the processor outputs the result of the classification by using a model acquired by producing teacher data through classification of a plurality of images obtained in time series through image pickup by the endoscope in accordance with an insertion state and performing learning with the teacher data by using a neural network.

8. The endoscope insertion control device according to claim 1, wherein the processor displays the result of the classification of the insertion state on a monitor.

9. The endoscope insertion control device according to claim 1, wherein
the processor selects an operation related to insertion of the endoscope,
the endoscope insertion control device further includes a recording unit configured to record the operation selected by the processor, and
the processor selects the next operation based on the operation recorded in the recording unit and the result of the classification of the insertion state.

10. The endoscope insertion control device according to claim 9, further comprising a database in which an operation related to insertion of the endoscope is registered based on a relation between the operation recorded in the recording unit and the result of the classification of the insertion state.

11. An endoscope insertion control method comprising:
acquiring a plurality of images related to insertion of an endoscope and obtained in time series;
classifying an insertion state of the endoscope based on the plurality of acquired images; and
selecting a next operation related to insertion of the endoscope by referring to a result of the classification,
wherein the selecting the next operation includes relaxing a forward movement condition when the insertion state is classified as the insertion state of forward movement.

12. A non-transitory recording medium in which a program is recorded, the program being configured to cause a computer to execute:
processing of acquiring a plurality of images related to insertion of an endoscope and obtained in time series;
processing of classifying an insertion state of the endoscope based on the plurality of images; and
processing of selecting a next operation related to insertion of the endoscope by referring to a result of the classification,
wherein the processing of selecting the next operation is processing of relaxing a forward movement condition when the insertion state is classified as the insertion state of forward movement.
